(19) United States
(12) Patent Application Publication
(10) Pub. No.: US 2019/0223959 A1
(43) Pub. Date: Jul. 25, 2019

(54) ELONGATED DEVICE WITH OPTICAL FIBER

(71) Applicant: KONINKLIJKE PHILIPS N.V., EINDHOVEN (NL)

(72) Inventor: Franciscus Reinier Antonius VAN DER LINDE, Schijndel (NL)

(21) Appl. No.: 16/327,405

(22) PCT Filed: Sep. 14, 2017
(86) PCT No.: PCT/EP2017/073135

§ 371 Date: Feb. 22, 2019

(30) Foreign Application Priority Data
Sep. 14, 2016 (EP) 16188759.1

(51) Int. Cl. A61B 34/20; G01H 9/00

(52) U.S. Cl. CPC A61B 34/20; A61B 2017/00526; G01H 9/004

(57) ABSTRACT

The present invention relates to an elongated device (10). The device (10) comprises an elongated body (12) having a proximal body end portion (14), a distal body end portion (16) and a longitudinal fiber lumen (18) extending from the proximal body end portion (14) to the distal body end portion (16). An optical fiber (20) extends through the fiber lumen (18) and has a distal fiber end portion (22). A socket (24) is arranged at the distal body end portion (16) of the elongated body (12), the socket (24) having a proximal socket end (28) and a distal socket end (30) and a hole (26) in axial alignment with the fiber lumen (18). The distal fiber end portion (22) is inserted in the hole (26), and the socket (24) is radio-opaque.

### FIELD OF THE INVENTION

The present invention generally relates to elongated devices which may be equipped with an optical fiber, in particular an optical fiber configured for optical shape sensing. The elongated devices may be medical interventional devices, like catheters, guidewires, endoscopes, stent delivery instruments, needles, and other minimally invasive devices.

### BACKGROUND OF THE INVENTION

Elongated medical devices, like catheters, guidewires, stent delivery systems, and other minimally invasive devices that are intended to be used in combination with X-ray guidance are radio-opaque over their entire length, or have radio-opaque markings at crucial positions, e.g. markerbands in balloon catheters and balloon-expandable stents to indicate the position of the balloon or the stent, to assess lengths or diameters with sizing catheters, or a tip position of the device, while these devices are being used under X-ray guidance.

Another technique for providing navigational guidance for elongated medical devices is optical shape sensing (OSS). An OSS enabled elongated device comprises an optical fiber as an integral part of the elongated device. OSS is a useful technology to reconstruct the three-dimensional shape of the elongated device intended for insertion into a patient's body. With OSS using an optical shape sensing fiber integrated in the elongated device, the three-dimensional shape of the device can be known and thus be made “visible” up to the tip of the device.

In OSS, an optical fiber integrated in the elongated device is interrogated by an interferometric distributed-sensing system that makes use of, e.g., Optical Frequency Domain Reflectometry (OFDR). In performing OSS, light from a light source, for example a tunable laser, is coupled into an optical fiber, and the reflected or backscattered light is made to interfere with light from the same light source that has traveled along a reference path. The optical response received from the optical fiber is correlated with the shape state of the optical fiber and, thus, of the elongated device, whereby the three-dimensional shape of the elongated device can be reconstructed in real time.

When using OSS, it is not always necessary to use X-ray to visualize the elongated device that is being used, since OSS itself can take care of the visualization of the elongated device. However, in some cases it is necessary to visualize the elongated device with X-rays, for example when the OSS coordinate domain needs to be registered to the X-ray coordinate domain. To do this registration, the elongated device with the integrated optical shape sensing fiber must also be visible for X-rays to perform this registration. The most important position of the entire elongated device is the tip. This part of the elongated device is the most distal part and thus most advanced in the body and must seek its way, or when it has to perform a task (e.g. visualization, treatment, measuring, or other tasks) it is typically positioned close to or at that end of the elongated device. For a proper registration of the tip to the X-ray coordinate domain, it is necessary that the distal end of the elongated device is well visible for X-rays, and, thus, should be radio-opaque at the distal end.

In order to achieve an optimal functionality of the OSS optical fiber, where it reconstructs the elongated device over the entire length thereof, it is desirable that the optical fiber is advanced in the elongated device all the way to the most distal end of the elongated device. For a proper shape reconstruction of the elongated device by OSS up to the tip of the device, it is necessary that the distal end of the optical fiber reaches to the distal end of the elongated device. The optical fiber extends through a fiber lumen in the body of the elongated device. When manufacturing the medical device, the optical fiber is inserted into the fiber lumen until the distal fiber end portion reaches the most distal end of the fiber lumen at or very close to the distal end of the elongated device. However, upon manufacturing the medical device, it is often difficult to keep the fiber lumen open to the optical fiber until the outermost distal end of the fiber lumen, because upon forming the tip of the elongated device, for example a catheter tip which is typically tapered or rounded, a thermal process is performed to shape the tip of the elongated device together with a mandrel to keep the fiber fiber lumen open. With this process step it can occur that the fiber lumen in the body of the elongated device gets a diameter at the distal end which is less than what is needed resulting in a situation that the optical fiber cannot be placed all the way to the distal end of the device.

US 2011/0098533 A1 discloses an endoscope comprising a distortion detection probe disposed in an insertion portion of the endoscope to be inserted into the interior of an examinee. The detection probe comprises an optical fiber provided with a plurality of fiber Bragg grating sensor sections that detect distortion of the insertion portion. The optical fiber extends to the distal end of the endoscope shaft and is fixed to a distal end rigid portion thereof via a holder which is a heat insulator or foam material.

WO 94/16623 discloses a catheter having an elongated body having a distal end adapted to be inserted into a body cavity. The catheter includes a fiber lumen extending through the elongated body. An optical fiber is disposed in the fiber lumen and extends from the distal end to the proximate thereof An X-ray sensitive phosphor material is disposed at a tip of the end of the optical fiber positioned at the distal end of the catheter.

U.S. Pat. No. 5,456,680 discloses a fiber optic catheter having a short guidewire lumen extending in a proximal direction from its distal end and an intermediate portion reinforced with a tapered mandrel which provides optimal steerability and trackability characteristics. The intermediate portion of the catheter may have a marker for visual identification, and the tip of the catheter may include a radio-opaque tip markerband for fluoroscopic identification.

US 2016/0101263 A1 discloses an instrument including an elongated, flexible body and a shape sensor including an optical fiber extending at least partially along the elongated, flexible body. The apparatus includes a radio-opaque material incorporated with the optical fiber at least partially along a length of the optical fiber.

### SUMMARY OF THE INVENTION

It is an object of the present invention to provide an elongated device which provides improved navigational guidance by optical shape sensing.

It is another object of the present invention to provide an elongated device which facilitates proper placement of a distal end of an optical fiber in the elongated device during manufacturing of the device.

According to an aspect of the invention, an elongated device is provided, comprising

an elongated body having a proximal body end portion, a distal body end portion and a longitudinal fiber lumen extending from the proximal body end portion to the distal body end portion,

an optical shape sensing fiber extending through the fiber lumen and having a distal fiber end portion,

a socket arranged at the distal body end portion of the elongated body, the socket having a proximal socket end and a distal socket end and a hole in axial alignment with the fiber lumen, wherein the distal fiber end portion is inserted in the hole, and the socket is radio-opaque so that the socket is visible by X-ray imaging.

The elongated device according to the invention uses a socket as a receiving part or receptacle for the distal fiber end portion of the optical fiber. The socket further is radio-opaque.

The socket is advantageous, because it may provide a well-defined position of the distal fiber end portion of the optical fiber at the distal body end portion of the device. During manufacturing, the socket may provide fiber lumen patency, because it may keep the fiber lumen open even if a thermal process is performed to shape the tip of the elongated device to form a determined tip shape, for example tapered or rounded. The socket preferably has a radial stiffness to resist to radial compression, and is further preferably resistant to deformation when heat is applied thereto, like in a thermal forming process. During manufacturing of the device, the body of the device may be provided with the socket in the desired place before inserting the optical fiber into the body fiber lumen.

The socket is further advantageous in that the socket ensures good “visibility” of the distal end of the elongated device for X-rays, because the socket is radio-opaque and arranged at the distal body end portion of the elongated body of the device. Thus, the socket ensures a more accurate registration of the distal optical fiber end to the elongated device in a X-ray coordinate domain. In particular, due to the radio-opacity of the socket, the distal body end portion of the elongated device may have a higher radio-opacity than the rest of the elongated body of the device, thus further improving registration of the distal optical fiber end to the elongated device.

The elongated device according to the invention may be a catheter, guidewire, endoscope or any other minimally invasive elongated device.

In a preferred embodiment, the hole of the socket may be open at the proximal socket end and closed at the distal socket end. During manufacturing, the closed distal end of the socket may provide a stop for the distal optical fiber end when the fiber is pushed through the fiber lumen into the socket, thus further facilitating the proper placement of the optical fiber end at the distal body end of the device.

In a further preferred embodiment, the distal fiber end portion of the optical fiber may reside in the hole in free floating fashion.

This embodiment is especially useful for elongated devices, which have a fiber lumen extending along a longitudinal center of the elongated body of the device. In this case, when the fiber lumen is in the center of the elongated body of the device, there is no translation of the fiber lumen when the elongated device is bent which results in the fact that the optical fiber stays in its position of the distal end of the fiber lumen without fixation means. This embodiment is advantageous in terms of manufacturing expenditure, because fixing or securing means like a glue or other fixing means are not necessary.

In an alternative embodiment, the distal fiber end portion of the optical fiber may be fixed to the socket in the hole.

This embodiment is especially useful for elongated devices, which have a fiber lumen which is off-axis with respect to the longitudinal center of the elongated body. In this case, when the fiber lumen is out of the center of the elongated body of the device, there is a translation of the fiber lumen when the elongated medical device is bent which results in the fact that the optical fiber tends not to stay in position. By fixation of the distal fiber end in the hole of the socket, it may be ensured that the distal fiber end portion is not moved away from its position in the socket due to bending of the device.

In a further preferred embodiment, an inner diameter at least of a proximal end of the hole may be equal to an inner diameter of the fiber lumen.

This measure has the advantage that the transition from the fiber lumen to the hole of the socket is smooth, i.e. free of any shoulders or edges. This in turn facilitates insertion of the optical fiber distal end portion into the hole of the socket during manufacturing.

In a further preferred embodiment, the hole may be cylindrical. This embodiment is advantageous in that the socket may be of simple design and, thus, can be easily manufactured with low cost.

In an alternative preferred embodiment, the hole may have a proximal portion tapering in direction from the proximal socket end to the distal socket end from a first inner diameter to a second inner diameter smaller than the first inner diameter, and a distal portion having the second inner diameter.

This embodiment is useful for elongated devices having an off-center fiber lumen as described above. In this case, the distal fiber end portion may be fixed in the distal portion of the socket, wherein the distal portion of the hole may have a cylindrical shape. While the tapering proximal portion of the socket may not only facilitate inserting of the distal fiber end portion into the hole, it may also protect the distal fiber end portion from breaking when the distal body end portion is bent during use of the device.

The taper angle of the proximal portion preferably is not too steep, just steep enough to give good guidance of the optical fiber from the fiber lumen into the socket. For example and preferably, the taper angle may be between 5 degrees and 15 degrees.

Further preferably, the second inner diameter of the socket according to the previous embodiment may be smaller than the inner diameter of the fiber lumen, thus providing a good self-fixation of the distal fiber end portion in the distal portion of the hole, even without additional fixing means like an adhesive.

In a further preferred embodiment, the socket may be embedded in the body. For example, if the elongated device is a catheter, the socket may be reflowed into the body, i.e. into the shaft of the catheter or its wall.

In an alternative preferred embodiment, the socket may be attached to an outermost distal body end of the body. This embodiment is useful for elongated devices which are too thin in cross-sectional diameter, for example guidewires for embedding the socket in the device body. In this case, where cross-sectional space inside the body of the elongated device is not sufficient for embedding, the socket may be soldered or glued to the distal end of the device body.

Preferably, the socket may have a short length, for example in the range of 0.4 mm to 1.5 mm. Thus, in comparison with the length of usual elongated devices like catheters or guidewires, the length of the socket may be very small. However, it is also possible to provide the socket with a longer length, e.g. of several centimeters. In this case, the socket should be more flexible in terms of bending so that it can be inserted into the fiber lumen over a longer length, while keeping that length of the device as flexible as the rest of the elongated device.

In a further preferred embodiment, the hole may have an inner diameter which is 0.01 mm to 0.1 mm, or 0.01 mm to 0.04 mm, or 0.05 mm to 0.1 mm larger than an outer diameter of the optical fiber.

Thus, in this way, the inner diameter of the hole is only slightly larger than the outer diameter of the optical fiber, thus ensuring a proper and well-defined positioning of the optical fiber in the socket during use of the device. In particular, movements of the distal fiber end portion away from the desired position can be excluded or at least be kept as small as possible.

Further, the socket may be stiff enough to resist to bending, or the socket may be flexible enough to be bent.

Example materials for a stiff socket are platinum/iridium alloy, gold or tungsten. A more flexible socket can be made from thermoplastic elastomers like Pebax™, Nylon, Polyurethane, or other typical thermoplastic elastomers used for catheters or guidewires mixed with high density materials, e.g. tungsten.

A flexible socket may also be manufactured from a material which is stiff per se, wherein flexibility of the socket however can be obtained by a structure with cutouts or by a coil structure of the socket. Such a structure with cutouts may be imparted by laser cutting. Further, it is also possible to add a coil structure via soldering, laser welding or gluing to a proximal side of a distal base portion of the socket.

The outer contour of the socket preferably has a cylindrical shape which is well suited for elongated devices having a body with a cylindrical basic shape.

### BRIEF DESCRIPTION OF THE DRAWINGS

These and other aspects of the invention will be apparent from and elucidated with reference to the embodiments described hereinafter. In the following drawings:

FIG. 1 shows an elongated device in a side view, partially interrupted and partially in longitudinal section;

FIGS. 2a) to 2c) show different views of a socket in enlarged scale for use in the device in FIG. 1, wherein FIG. 2a) shows a proximal front end view, FIG. 2b) a side view, and FIG. 2c) a distal front end view;

FIG. 3 shows the socket in FIGS. 2a) to 2c) in a longitudinal section;

FIG. 4 shows a further embodiment of a socket in enlarged scale in a longitudinal section for use in the device in FIG. 1;

FIG. 5 shows a further embodiment of a socket in enlarged scale in a longitudinal section for use in the device in FIG. 1;

FIG. 6 shows a further embodiment of a socket in enlarged scale in a side view for use in the device in FIG. 1;

FIG. 7 shows an embodiment of portion A of the device in FIG. 1 in longitudinal section and in an enlarged scale with respect to FIG. 1;

FIG. 8 shows another embodiment of portion A of the device in FIG. 1 in longitudinal section and in an enlarged scale with respect to FIG. 1; and

FIG. 9 shows still another embodiment of portion A of the device in FIG. 1 longitudinal section and in an enlarged scale with respect to FIG. 1.

### DETAILED DESCRIPTION OF THE INVENTION

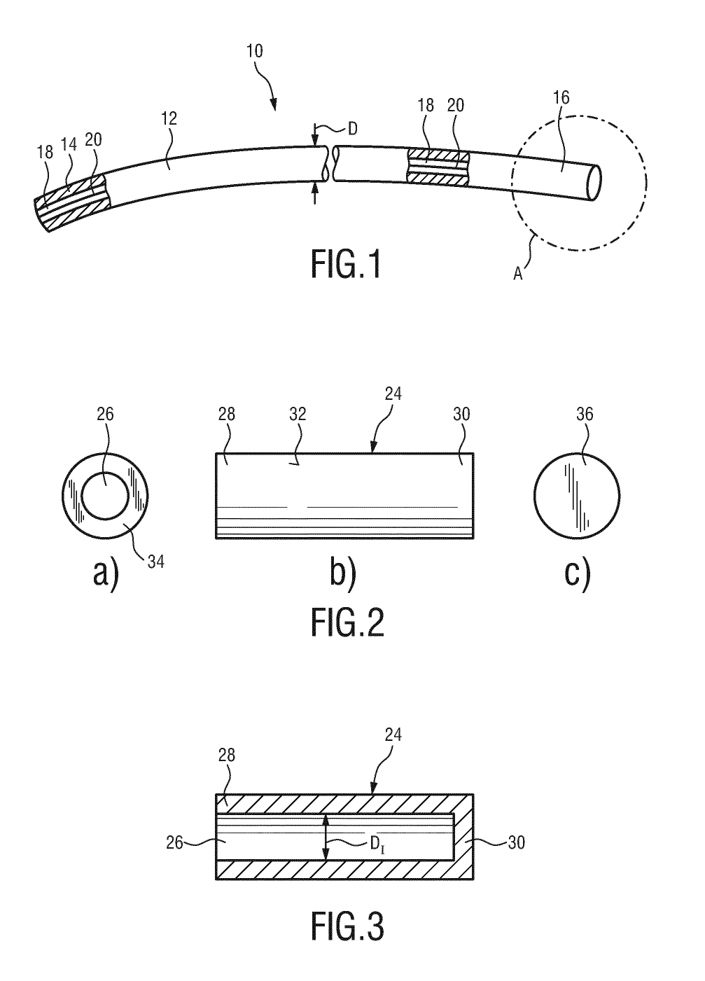

FIG. 1 shows an elongated device labeled with reference numeral 10. The elongated device 10 may have a length which is much larger than its diameter D. For example, the elongated device 10 may have a length which is larger than 1 or 2 m. The diameter D may be as small as a few millimeters, for example may be in a range of about 1 to 3 mm. The elongated device 10 is flexible and, thus, may be bent. The elongated device 10 may be, for example, a catheter, a guidewire, an endoscope, a needle, a stylet or any other minimally invasive device.

The elongated device 10 comprises an elongated body 12 having a proximal body end portion 14 and a distal body end portion 16.

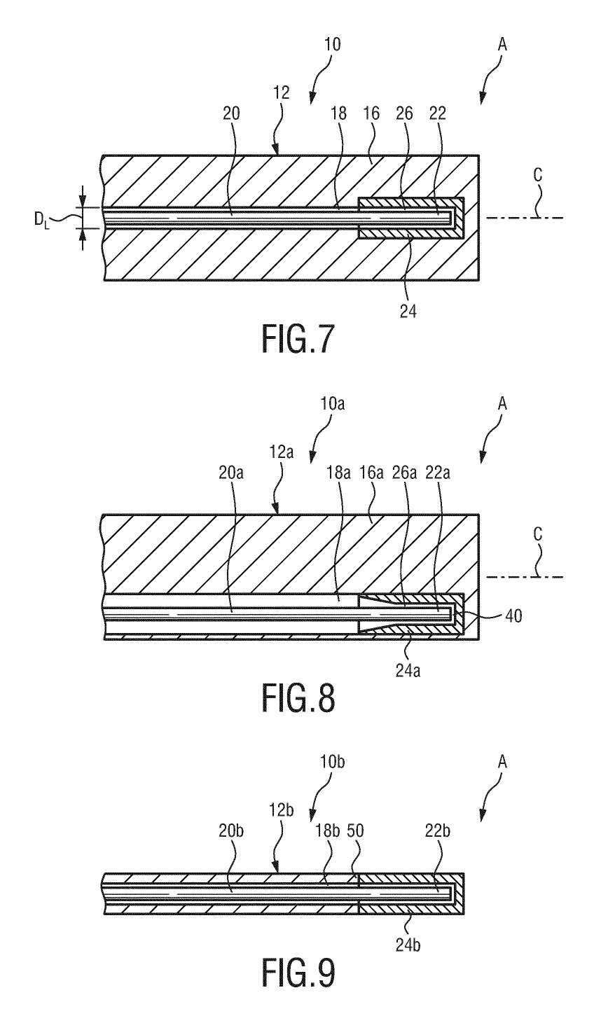

A longitudinal fiber lumen 18 extends from the proximal body end portion 14 to the distal body end portion 16, e.g. as shown in FIG. 7.

An optical fiber 20 extends through the fiber lumen 18 from the proximal body end portion 14 until the distal body end portion 16 of the elongated body 12, e.g. as shown in FIG. 7. The optical fiber 20 has a distal fiber end portion 22 (FIG. 7). The optical fiber 18 is configured for optical shape sensing (OSS). To this end, the optical fiber 20 may be connectable, on the proximal side, to an interrogator console (not shown), as for example known in the art. With OSS using the optical fiber 20, the three-dimensional shape of the optical fiber 20 along the length of the elongated device 10 and, thus, the three-dimensional shape of the elongated device 10 may be reconstructed. To this end, the optical fiber 20 may be interrogated by an interferometric distributed-sensing system comprised in the interrogator console, that may make use of, e.g., Optical Frequency Domain Reflectometry. When light from a light source, for example a tunable light source, is coupled into the optical fiber 20, the reflected or backscattered light is made to interfere with light from the same light source that has traveled along a reference path. The optical response received from the fiber 20 is influenced by strain in the fiber 20 when the fiber 20 is bent, and from the optical response the 3D-shape of the fiber 20 and, thus the device 10 may be reconstructed.

The inner diameter of the fiber lumen 18 may be only slightly larger than the outer diameter of the optical fiber 20.

For proper OSS results, it is desirable that the optical fiber 20 extends as much as possible over the entire length of the elongated device 10. In particular, it is desirable that the distal fiber end portion 22 is arranged in or at the distal body end portion 16 of the elongated body 12 of the device 10.

In order to ensure proper positioning of the distal fiber end portion 22 of the optical fiber 20 as close as possible to or even better at the distal body end portion 16, a socket 24 as shown in FIG. 7 is arranged at the distal body end portion 16 of the elongated body 12.

With reference to FIGS. 2a) to 2c), FIG. 3 and FIG. 7, the socket 24 has a hole 26 in alignment with the fiber lumen 18 (FIG. 7). The socket 24 serves as a receiving part for the distal fiber end portion 22 of the optical fiber 20, i.e. the distal fiber end portion 22 is inserted in the hole 26 of the socket 24.

The hole 26 may be open at a proximal socket end 28 and closed at a distal socket end 30. In this way, the hole 26 is a blind hole. The distal fiber end portion 22 can be inserted through the open proximal socket end 28 into the hole 26 until it may abut against the closed distal socket end 30, as shown in FIG. 7.

An outer surface 32 of the socket 24 may be cylindrical in shape. FIG. 2a) shows a proximal front end surface 34 of the socket 24, and FIG. 2c) shows a distal front end surface 36 of the socket 24.

The socket 24 is radio-opaque so that it is well visible by X-ray imaging. The socket 24 may be radio-opaque as a whole, or may be radio-opaque at least in one or more portions of the socket 24.

In the embodiment of the socket 24 according to FIG. 3 and FIG. 7, the hole 26 has an overall cylindrical shape. Such a socket 24 is useful when the fiber lumen 18 is arranged in the longitudinal center C (FIG. 7) of the elongated body 12 as shown in FIG. 7. Further, in this embodiment, the distal fiber end portion 22 may be received in the hole 26 in free floating fashion. A fixation of the fiber end portion 22 is not necessary due to the fact that when the elongated body 12 is bent, the optical fiber 20 does not experience a relative change of length with respect to the center C of the body 12.

An inner diameter D_{I} of the hole 26 may be equal to an inner diameter D_{L} of the fiber lumen 18, and because the hole 26 is aligned with the fiber lumen 18 the transition from the fiber lumen 18 to the hole 26 is as smooth as possible.

The inner diameter of the hole 26 may be 0.05 mm to 0.10 mm larger than the outer diameter of the optical fiber 20 in order to obtain a free floating reception of the distal fiber end portion 22 in the hole 26.

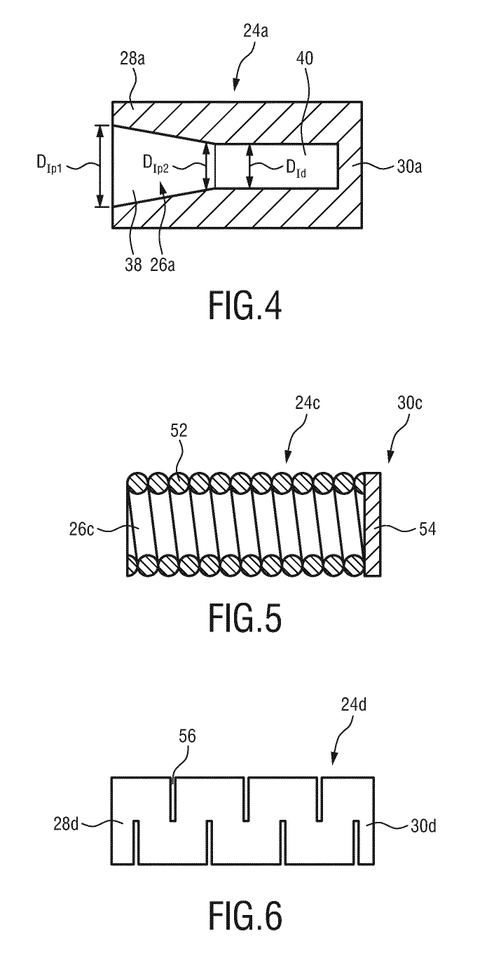

With reference to FIGS. 4 and 8, another embodiment of a socket 24a and its installation in a distal body end portion 16a of an elongated body 12a of an elongated device 10a will be described. While the socket 24a has an outer cylindrical shape like the socket 24 described before, a hole 26a which is open at a proximal socket end 28a and closed at a distal socket end 30a is shaped differently from the hole 26 of the socket 24.

The hole 26a of the socket 24a has a proximal portion 38 and a distal portion 40. The distal portion 40 has an inner diameter D_{Id} which is constant over the length of the distal portion 40 like the inner diameter D_{I} of the hole 26 of the socket 24. The proximal portion 38 instead tapers in direction from the proximal socket end 28a to the distal socket end 30a from a first inner diameter D_{Ip1} to a second inner diameter D_{Ip2} smaller than the first inner diameter D_{Ip1}, wherein the second inner diameter D_{Ip2} is equal to the inner diameter D_{Id} of the distal portion 40 of the hole 26a.

This embodiment of the socket 24a is useful when the fiber lumen 18a as shown in FIG. 8 is off-axis with respect to the center C of the elongated body 12a of the device 10a. In this case, when the elongated body 12a is bent, the optical fiber 20a may experience a relative change of length with respect to the center C of the elongated body 12a which can move the optical fiber end 22a with respect to the socket 24a. Therefore, in this embodiment it is preferred if the distal fiber end portion 22a is fixed to the socket 24a in the hole 26a, wherein it is preferred if only that portion of the distal fiber end portion 22a is fixed to the socket 24a which is received in the distal portion 40 of the hole 26a. This may be achieved in that the inner diameter D_{I}d of the distal portion 40 of the hole 26a may be chosen to be just a little larger than the outer diameter of the optical fiber 20a. Preferably, the inner diameter D_{I}d may be 0.010 to 0.040 mm larger than the outer diameter of the optical fiber 20a. The inner diameter D_{Id} of the distal portion 40 may be smaller than the inner diameter of the fiber lumen 18a.

The taper angle of the proximal portion 38 of the hole 26a is not too steep, but just enough to give good guidance of the optical fiber 20a from the fiber lumen 18a into the socket 24a. For example, the taper angle may be between 5 degrees and 15 degrees.

The length of the distal portion 40 of the hole 26a may be between 0.2 and 0.8 mm, while the length of the proximal portion 38 of the hole 26a may be between 0.2 mm and 0.3 mm.

In the embodiments shown in FIGS. 7 and 8, the socket 24 or the socket 24a, respectively, are embedded in the elongated body 12 in the distal body end portion 16. This can be done by reflowing the socket 24 or 24a into the material or wall of the body 12. Embedding may be envisaged in case the elongated device 10 is a catheter or similar device.

In case of thinner elongated devices, for example guidewires, and e.g. as shown in FIG. 9, a socket 24b for receiving a distal fiber end portion 22b of an optical fiber 20b extending through a fiber lumen 18b of the body 12b may be attached to an outermost distal body end 50 of the body 12b. The socket 24b may be of the same construction as the socket 24 in FIGS. 3 and 7. Attaching the socket 24b at the distal end 50 of the body 12b may be accomplished by soldering, gluing and the like.

In the embodiments according to FIGS. 3, 4, 7, 8, the socket 24 or the socket 24a may have a very short length, for example in the range of 0.4 mm to 1.5 mm.

The sockets 24 and 24a may be stiff enough to resist to bending in longitudinal direction. Materials for the sockets 24 and 24a which provide high stiffness may be platinum/iridium alloy, gold or tungsten which also provide good radio-opacity.

If the sockets 24 and 24a should be more flexible, they can be made from thermoplastic elastomers like Pebax™, Nylon, Polyurethane, or other typical thermoplastic elastomers as used for example for catheters or guidewires mixed with high density materials, e.g. tungsten.

Nevertheless, it is also possible to provide a socket which is longer in length and which are more flexible to be able to bend. Such embodiments are shown in FIGS. 5 and 6.

FIG. 5 shows an embodiment, where a socket 24c comprises a coil 52 with a plurality of windings, similar to a helix spring. A distal base plate 54 closes the hole 26c of the socket 24c at the distal socket end 30c.

The socket 24c may be installed in the body 12 of the elongated device 10 instead of the socket 24 as shown in FIG. 7. The length of the socket 24c may be longer than the length of the socket 24, for example longer than 1.5 mm, for example up to several centimeters, for example up to 5 cm, or even up to 10 cm.

The coil 52 may be made of a material which is stiff per se like the metals or alloys mentioned above, or may be made from a material which is more flexible per se, for example a thermoplastic elastomer.

FIG. 6 shows another embodiment of a socket 24d having a closed distal socket end 30d and an open proximal socket end 28d. The socket 24d may have a length of several centimeters. In order to obtain a higher degree of flexibility of the socket 24d, cutouts 56 are made into the wall of the socket 24d, for example by laser cutting. The structure of the cutouts 56 as shown in FIG. 6 is only one example of a plurality of possible structures. Also in this case, the socket 24d may be made of a material which is stiff per se, while the flexibility is obtained by the cutouts 56 in the socket wall.

The sockets 24, 24a, 24b, 24c, 24d described above provide sufficient radial stiffness to keep the lumen 18 in the body 12 of the elongated device 10 during manufacturing and use of the device 10 open.

Due to the radio-opacity of the sockets described herein, the distal end of the elongated device 10 may be made visible under X-ray imaging, and because the sockets described herein may ensure that the fiber distal end of the optical fiber is properly positioned as close as possible to the distal end of the elongated device 10, proper registration of the OSS data to the elongated device in the X-ray coordinate system may be ensured.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. Elongated device, comprising:
an elongated body having a proximal body end portion, a distal body end portion and a longitudinal fiber lumen extending from the proximal body end portion to the distal body end portion,
an optical shape sensing fiber extending through the fiber lumen and having a distal fiber end portion,
a socket arranged at the distal body end portion of the elongated body, the socket having a proximal socket end and a distal socket end and a hole in axial alignment with the fiber lumen, wherein the distal fiber end portion is inserted in the hole, and the socket is radio-opaque so that the socket is visible by X-ray imaging.

2. Device of claim 1, wherein the hole of the socket is open at the proximal socket end and closed at the distal socket end.

3. Device of claim 1, wherein the distal fiber end portion resides in the hole of the socket in free floating fashion.

4. Device of claim 1, wherein the distal fiber end portion is fixed to the socket in the hole.

5. Device of claim 1, wherein an inner diameter at least of a proximal end of the hole is equal to an inner diameter of the fiber lumen.

6. Device of claim 1, wherein the hole is cylindrical.

7. Device of claim 1, wherein the hole has a proximal portion tapering in direction from the proximal socket end to the distal socket end from a first inner diameter to a second inner diameter smaller than the first inner diameter, and a distal portion having the second inner diameter.

8. Device of claim 7, wherein the second inner diameter is equal to the inner diameter of the fiber lumen.

9. Device of claim 1, wherein the socket is embedded in the body.

10. Device of claim 1, wherein the socket is attached to an outermost distal body end of the body.

11. Device of claim 1, wherein the hole has an inner diameter which is 0.01 mm to 0.1 mm larger than an outer diameter of the optical fiber.

12. Device of claim 1, wherein the socket is stiff enough to resist to bending.

13. Device of claim 1, wherein the socket is flexible enough to be bent.

14. Device of claim 13, wherein the socket comprises a structure with cutouts or comprises a coil structure.
